(19) United States
(12) Patent Application Publication
(10) Pub. No.: US 2021/0393002 A1
(43) Pub. Date: Dec. 23, 2021

(54) WRISTBAND WITH A BUILT-IN CONTAINER

(71) Applicant: David Ascencio, Oakland, CA (US)

(72) Inventor: David Ascencio, Oakland, CA (US)

(21) Appl. No.: 16/946,392

(22) Filed: Jun. 19, 2020

(51) Int. Cl. A44C 5/00; A44C 15/00; A47K 5/122; A61L 2/18

(52) U.S. Cl. CPC A44C 5/003; A44C 15/002; B05B 9/08; A61L 2/18; A47K 5/122

(57) ABSTRACT

A wristband-container combination “Saniband” that secures to the wrist of a user so as to provide an accessible cavity for dispensing fluids or solutions retained therein.

### BACKGROUND OF THE INVENTION

The present invention relates to wristbands and containers and, more particularly, to a wristband with a built-in container for holding soluble solutions for dispensing.

It is very common that from time to time when an individual is on the go, they need to access a fluid. The fluid may be a sanitizer, lotion, ointment, liquid or viscous medicament, any soluble solution. As a result, it is not uncommon for a need to have such fluids handy and readily accessible even though the individual may be inconveniently far away from where the normally store said fluid.

Small bottles are the typical solution for storing and dispensing said fluids; however, small bottles or other containers get lost in purses and bags or misplaced somewhere, and so are not at hand when needed. In short, small containers and bottles are from time to time no accessible when needed, while other current solutions use more space and time to find them because of their ‘loose’, portable nature. Case in point, if a person is driving or jogging it can be a challenge to find and use a bottle of solution, as it is not easily accessible.

As can be seen, there is a need for a wristband with a built-in container for holding soluble solutions for dispensing. The solution may be embodied in a wristband-container combination that includes a port for both dispensing and refilling the soluble solution. The wristband-container combination disclosed in the present invention is colloquially known as “Saniband.” Saniband is a waterproof and durable innovation that can solve the problems of the prior art.

The present invention solves the above-mentioned problem by always being at hand and easily accessible when the wearer of the wristband is in need of a fluid or solution such as sanitizer, lotion or ointment; thereby saving time and space. In sum, the present invention embodies a waterproof, reusable, refillable container formed into a wristband to aid in easy access and convenience of fluids stored therein.

### SUMMARY OF THE INVENTION

In one aspect of the present invention, a wristband-container combination, includes the following: a wristband; a cavity embedded in the wristband; a port fluidly coupled to the cavity; and a push indentation disposed on an external surface of the cavity.

In another aspect of the present invention, wristband-container combination, includes the following: a wristband; a cavity embedded in the wristband, wherein the cavity is dimensioned to provide a volume between 0.25 to 0.50 fluid ounces; a port fluidly coupled to the cavity, wherein the port is a tubular protrusion from the external surface of the cavity; a push indentation disposed on an external surface of the cavity; a plug dimensioned to be received in the tubular protrusion; a grip flange extending radially from a distal end of the plug; and a plug attachment interconnecting the wristband and the plug.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a top view of an exemplary embodiment of the present invention, shown in use;

FIG. 3 is a section view of an exemplary embodiment of the present invention, taken along line 3-3 from FIG. 1;

FIG. 4 is a section view of an exemplary embodiment of the present invention, taken along line 4-4 from FIG. 2;

FIG. 5 is a detailed section view of an exemplary embodiment of a port and plug of the present invention; and

FIG. 6 is a detailed section view of an exemplary embodiment the a port and plug of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a wristband-container combination that secures to the wrist of a user so as to provide an accessible cavity for dispensing fluids or solutions retained therein.

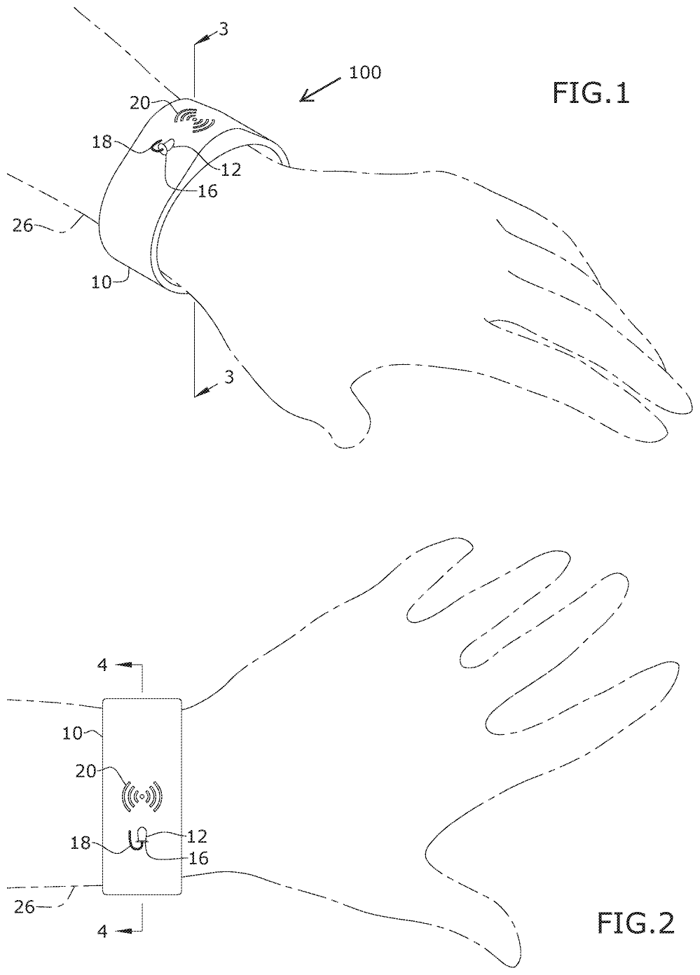

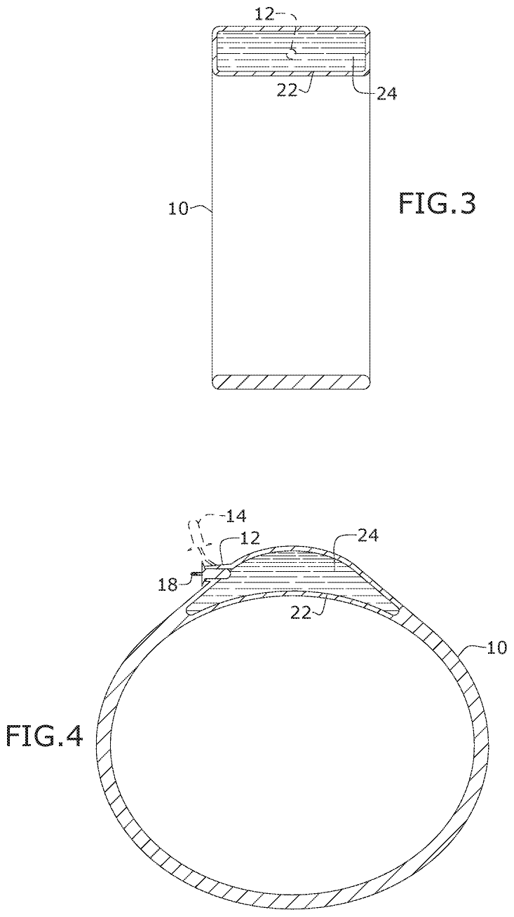

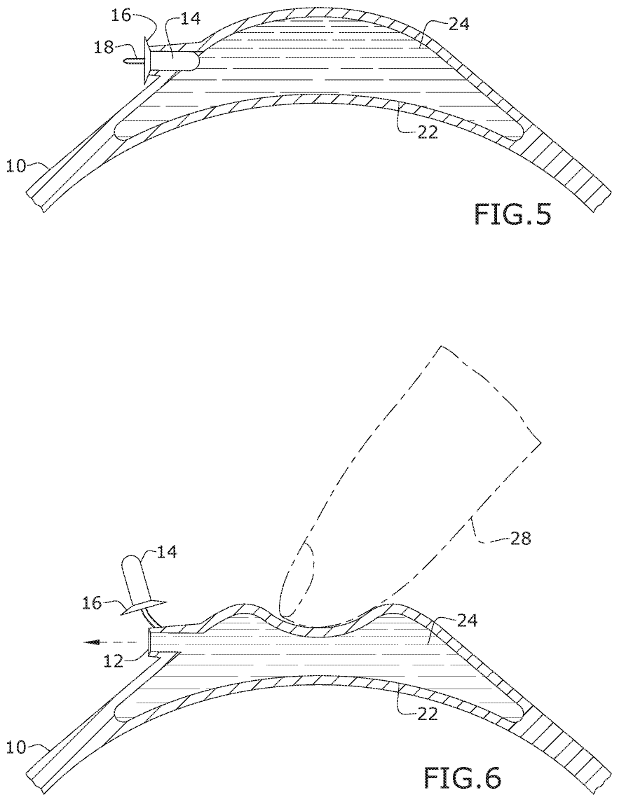

Referring to FIGS. 1 through 6, the present invention may include a wristband-container combination 100 having a wristband 10 dimensioned and adapted to wrap around and be secured to a wrist 26 of a user. The wristband 10 embodies a cavity 22 for retrievably storing a fluid 24. The cavity 22 fluidly connects to outside the wristband 10 by way of a port 12. The above-mentioned components may be made from silicone, various plasticized materials, or other material that have elastic, stretchable properties exhibiting temporary deformation so that that component may be recoverable as a deformation disappears after the removal of applied forces that caused the deformation.

The cavity 22 may be a built-in roundish pouch on top of the wristband 10. The cavity 22 may be able to hold at least 0.25 to 0.50 oz of soluble solution/fluid 24. The port 12 may be a tubular protrusion. The port 12 is dimensioned and adapted to dispense the fluid 24 from and refill the fluid 24 into the cavity 22. A plug 14 may be dimensioned and adapted to be slidably received in the tubular port 12 so as to fluidly disconnect the cavity 22 and the outside of the wristband 10; or in other words, moving the port 12 between an open and a closed condition with the plug 14 slidably received in the port 12. A distal end of the plug 14 may provide a grip 16 extending generally radially from the body of the plug 14, as illustrated in FIG. 5. The grip 16 can be a flange or other structure adapted to facilitate manipulating the plug 12 between the open and closed conditions. A plug attachment 18 may be extend outwardly from the grip 16. The plug attachment 18 may interconnect the plug 14 and the wristband 10 so that the plug 14 may be removed from the port 12, moving the cavity 22 to an open condition, yet the plug 14 will not be disconnected from the wristband.

Along an upper portion of the wristband-container combination 100 adjacent the cavity 22 may be a push indentation 20. The push indentation 20 may facilitate or enable a user, typically by way of their finger 28, to pressure or urge fluid 24 out of the cavity, as illustrated in FIG. 5. The push indentation 20 may a top portion of the cavity 22 having a bulbous or nipple-like shape.

The wristband-container combination 100 can be customized with different colors and designs. The wristband-container combination 100 may be made from flexible silicone rubber which can be extruded into strips or tubes of varying thickness, then compression molded into a round bracelet shape. Color dyeing is usually a pre-molding process.

The size and shape of the wristband-container combination 100 should be ergonomic and easy to use. Degree and position of the squirt port 12 can vary from wristband-container combination 100 to wristband-container combination 100.

A method of using the present invention may include the following. The wristband-container combination 100 disclosed above may be provided. A wearer may wrap the wristband 10 about their wrist 26. The user may remove the plug 14, moving the port 12 to the open condition, and then by applying pressure—for instance, through depressing the push indentation 20 with their finger 28—the user can urge the fluid stored in the cavity 22 out through the port 12.

For instance, a healthcare worker wearing the wristband-container combination 100 would not need to reach in their pockets or search for their bottle of sanitizer all the time—the fluid 24 would be on hand when needed for lessening the risk of spread of bacteria and viruses on one's self and so from person to person. The wristband-container combination 100 would aid in lessening the spread of the current Covid-19 virus and other infectious diseases by people continuing to sanitize their hands through having sanitizer ready for use wrapped around their wrist 28.

Additionally, the present invention could be used in medical and sports field for lessening the spread of bacteria and viruses.

It should be understood, of course, that the foregoing relates to exemplary embodiments of the invention and that modifications may be made without departing from the spirit and scope of the invention as set forth in the following claims.

What is claimed is:

1. A wristband-container combination, comprising:
a wristband;
a cavity embedded in the wristband;
a port fluidly coupled to the cavity; and
a push indentation disposed on an external surface of the cavity.

2. The wristband-container combination of claim 1, wherein the port is a tubular protrusion from the external surface of the cavity.

3. The wristband-container combination of claim 2, further comprising a plug dimensioned to be received in the tubular protrusion.

4. The wristband-container combination of claim 3, further comprising a grip flange extending radially from a distal end of the plug.

5. The wristband-container combination of claim 2, further comprising a plug attachment interconnecting the wristband and the plug.

6. The wristband-container combination of claim 1, wherein the cavity is dimensioned to provide a volume between 0.25 to 0.50 fluid ounces.

7. A wristband-container combination, comprising:
a wristband;
a cavity embedded in the wristband, wherein the cavity is dimensioned to provide a volume between 0.25 to 0.50 fluid ounces;
a port fluidly coupled to the cavity, wherein the port is a tubular protrusion from the external surface of the cavity;
a push indentation disposed on an external surface of the cavity;
a plug dimensioned to be received in the tubular protrusion;
a grip flange extending radially from a distal end of the plug; and
a plug attachment interconnecting the wristband and the plug.
